(19) United States
(12) Patent Application Publication
(10) Pub. No.: US 2016/0278644 A1
(43) Pub. Date: Sep. 29, 2016

(54) CONTACT-LESS BLOOD PRESSURE MEASUREMENT

(71) Applicant: Quanttus, Inc., Cambridge, MA (US)

(72) Inventor: David Da He, Cambridge, MA (US)

(21) Appl. No.: 15/079,817

(22) Filed: Mar. 24, 2016

Related U.S. Application Data
(60) Provisional application No. 62/138,079, filed on Mar. 25, 2015.

(51) Int. Cl. A61B 5/021; A61B 5/00; A61B 5/11

(52) U.S. Cl. CPC A61B 5/02125; A61B 5/1102; A61B 5/7275

(57) ABSTRACT

The technology described in this document can be embodied in a method that includes receiving optical data including information associated with a subject, and determining from the optical data, a first dataset and a second dataset. The first dataset represents time-varying color change at a first body part of the subject, and the second dataset represents time-varying characteristics at a second body part of the subject. The method includes identifying a first point in the first dataset, and a second point in the second dataset. The first point represents a time at which a pulse pressure wave traverses the first body part of the subject, and the second point represents a time at which the pulse pressure wave traverses the second body part of the subject. A pulse transit time (PTT) between the first and second body parts can be calculated as a difference between the first and second points.

### PRIORITY CLAIM

This application claims priority to U.S. Provisional Application No. 62/138,079, filed on Mar. 25, 2015, the entire content of which is incorporated herein by reference.

### TECHNICAL FIELD

This document describes technology related to devices for health and fitness monitoring.

### BACKGROUND

Various types of sensors can be used in wearable devices. Wearable devices including sensors are currently used for various purposes, including monitoring a user's physical activity.

### SUMMARY

In one aspect, this document features a computer implemented method that includes receiving optical data including information associated with a subject. The method also includes determining from the optical data, a first dataset and a second dataset. The first dataset represents time-varying color change at a first body part of the subject, and the second dataset represents time-varying characteristics at a second body part of the subject. The method further includes identifying a first point in the first dataset, and a second point in the second dataset. The first point represents a time at which a pulse pressure wave traverses the first body part of the subject, and the second point represents a time at which the pulse pressure wave traverses the second body part of the subject. The method also includes computing a pulse transit time (PTT) as a difference between the first and second points. The PTT represents a time taken by the pulse pressure wave to travel from the second body part to the first body part of the subject.

In another aspect, the document features a computer-implemented method that includes receiving a plurality of frames of video data featuring a subject, and determining from the video data, a first dataset representing time-varying motion at a first body part of the subject. The method also includes determining blood pressure of the subject based on the first dataset. The blood pressure can be determined as a function of a pulse transit time (PTT) that represents a time taken by a pulse pressure wave to travel from a second body part to the first body part of the subject. Computing the PTT can include determining a second dataset representing time-varying color change at the first body part of the subject, and identifying a first point in the second dataset, the first point representing an arrival time of the pulse pressure wave at the first body part of the subject. Computing the PTT can also include identifying a second point in the first dataset, the second point representing an earlier time at which the pulse pressure wave traverses the second body part of the subject, and computing the pulse transit time (PTT) as a difference between the first and second points.

In another aspect, the document features a computer-implemented method that includes receiving a plurality of frames of video data featuring a subject, determining from the video data, a first dataset representing time-varying skin tone change at a first body part of the subject, and determining blood pressure of the subject based on the first dataset. The blood pressure can be determined as a function of a pulse transit time (PTT) that represents a time taken by a pulse pressure wave to travel from a second body part to the first body part of the subject. Computing the PTT can include determining from the video data, a second dataset representing time-varying motion at the first body part of the subject, and identifying a first point in the first dataset, the first point representing an arrival time of the pulse pressure wave at the first body part of the subject. Computing the PTT can also include identifying a second point in the second dataset, the second point representing an earlier time at which the pulse pressure wave traverses the second body part of the subject, and computing the pulse transit time (PTT) as a difference between the first and second points.

In another aspect, the document features a system that includes a memory and one or more processing devices, wherein the one or more processing devices are configured to receive optical data including information associated with a subject, determine, from the optical data, a first dataset representing time-varying color change at a first body part of the subject, and determine, from the optical data, a second dataset representing time-varying characteristics at a second body part of the subject. The one or more processing devices are further configured to identify a first point in the first dataset, the first point representing a time at which a pulse pressure wave traverses the first body part of the subject, and identify a second point in the second dataset, the second point representing a time at which the pulse pressure wave traverses the second body part of the subject. The one or more processing devices are also configured to compute a pulse transit time (PTT) as a difference between the first and second points, the PTT representing a time taken by the pulse pressure wave to travel from the second body part to the first body part of the subject.

In another aspect, the document features a system that includes memory, and one or more processing devices, wherein the one or more processing devices are configured to receive a plurality of frames of video data featuring a subject, determine from the video data, a first dataset representing time-varying motion at a first body part of the subject, and determine blood pressure of the subject based on the first dataset. The blood pressure can be determined as a function of a pulse transit time (PTT) that represents a time taken by a pulse pressure wave to travel from a second body part to the first body part of the subject. Computing the PTT can include determining a second dataset representing time-varying color change at the first body part of the subject, and identifying a first point in the second dataset, the first point representing an arrival time of the pulse pressure wave at the first body part of the subject. Computing the PTT also includes identifying a second point in the first dataset, the second point representing an earlier time at which the pulse pressure wave traverses the second body part of the subject, and computing the pulse transit time (PTT) as a difference between the first and second points.

In another aspect, the document features a system that includes memory and one or more processing devices, wherein the one or more processing devices are configured to receive a plurality of frames of video data featuring a subject, determine from the video data, a first dataset representing time-varying skin tone change at a first body part of the subject, and determine blood pressure of the subject based on the first dataset. The blood pressure can be determined as a function of a pulse transit time (PTT) that represents a time taken by a pulse pressure wave to travel from a second body part to the first body part of the subject. Computing the PTT can include determining from the video data, a second dataset representing time-varying motion at the first body part of the subject, and identifying a first point in the first dataset, the first point representing an arrival time of the pulse pressure wave at the first body part of the subject. Computing the PTT can also include identifying a second point in the second dataset, the second point representing an earlier time at which the pulse pressure wave traverses the second body part of the subject, and computing the pulse transit time (PTT) as a difference between the first and second points.

In another aspect, the document features one or more machine-readable storage devices storing instructions that, upon execution by one or more processing devices, cause the one or more processing devices to perform various operations that include receiving optical data including information associated with a subject. The operations also include determining from the optical data, a first dataset and a second dataset. The first dataset represents time-varying color change at a first body part of the subject, and the second dataset represents time-varying characteristics at a second body part of the subject. The operations further include identifying a first point in the first dataset, and a second point in the second dataset. The first point represents a time at which a pulse pressure wave traverses the first body part of the subject, and the second point represents a time at which the pulse pressure wave traverses the second body part of the subject. The operations also include computing a pulse transit time (PTT) as a difference between the first and second points. The PTT represents a time taken by the pulse pressure wave to travel from the second body part to the first body part of the subject.

In another aspect, the document features one or more machine-readable storage devices storing instructions that, upon execution by one or more processing devices, cause the one or more processing devices to perform various operations that include receiving a plurality of frames of video data featuring a subject, and determining from the video data, a first dataset representing time-varying motion at a first body part of the subject. The operations also include determining blood pressure of the subject based on the first dataset. The blood pressure can be determined as a function of a pulse transit time (PTT) that represents a time taken by a pulse pressure wave to travel from a second body part to the first body part of the subject. Computing the PTT can include determining a second dataset representing time-varying color change at the first body part of the subject, and identifying a first point in the second dataset, the first point representing an arrival time of the pulse pressure wave at the first body part of the subject. Computing the PTT can also include identifying a second point in the first dataset, the second point representing an earlier time at which the pulse pressure wave traverses the second body part of the subject, and computing the pulse transit time (PTT) as a difference between the first and second points.

In another aspect, the document features one or more machine-readable storage devices storing instructions that, upon execution by one or more processing devices, cause the one or more processing devices to perform various operations that include receiving a plurality of frames of video data featuring a subject, determining from the video data, a first dataset representing time-varying skin tone change at a first body part of the subject, and determining blood pressure of the subject based on the first dataset. The blood pressure can be determined as a function of a pulse transit time (PTT) that represents a time taken by a pulse pressure wave to travel from a second body part to the first body part of the subject. Computing the PTT can include determining from the video data, a second dataset representing time-varying motion at the first body part of the subject, and identifying a first point in the first dataset, the first point representing an arrival time of the pulse pressure wave at the first body part of the subject. Computing the PTT can also include identifying a second point in the second dataset, the second point representing an earlier time at which the pulse pressure wave traverses the second body part of the subject, and computing the pulse transit time (PTT) as a difference between the first and second points.

Implementations of the above aspects can include one or more of the following features.

The second body part can include at least a portion of the first body part. The time varying characteristics can include motion of the second body part. The time varying characteristics at the second body part can include time-varying color change. The optical data can include video data that includes a plurality of frames featuring the subject. Corresponding portions in the plurality of frames can be identified, wherein the corresponding portions represent the first body part at different points in time. The plurality of frames can be captured by a single camera. Determining the first dataset can include selecting from the video data, a set of one or more pixels that represents at least a portion of the first body part, and determining the first dataset as a time-varying average of pixel values in the set. The set of one or more pixels can be manually selected via a user-interface. The time-varying average can be computed based on a particular color component of the pixel values in the set. The color component can be selected based on a nature of ambient light in which the video data is captured. The color component can be selected based on a nature of skin color of the subject. Determining the second dataset can include selecting a set of one or more reference points in the optical data representing the second body part, and tracking a motion of the selected set of one or more reference points along a particular direction to determine the second dataset. The second dataset can include ballistocardiogram (BCG) data. The second dataset can be filtered to obtain the BCG data. The second dataset can be filtered using a filter having a passband within a frequency range of 0 and 30 Hz. Identifying the first point can include computing a cross-correlation of a template segment with each of multiple segments of the first dataset, identifying, based on the computed cross-correlations, at least one candidate segment of the first dataset as including the first point, and identifying a first local maximum or minimum, or zero-crossing within the identified candidate segment as the first point. Identifying the second point can include computing a cross-correlation of a template segment with each of multiple segments of the second dataset, identifying, based on the computed cross-correlations, at least one candidate segment of the second dataset as including the second point, and identifying a first local maximum or minimum, or zero-crossing within the identified candidate segment as the second point. The blood pressure of the subject can be computed as a function of the PTT. The blood pressure can include a systolic pressure and a diastolic pressure. The diastolic pressure can be calculated as a linear function of the logarithm of the PTT. The method systolic pressure can be calculated as a function of the diastolic pressure. Determining the first or second dataset can include identifying at least one of the first body part and the second body part from the video data. The PTT can be computed responsive to a user-input requesting measurement of a vital sign of the subject.

Particular implementations may realize one, or more of the following advantages. Blood pressure and other health related parameters can be computed based on contact-less and non-invasive measurements. Motion data (e.g., ballistocardiogram (BCG) or motioncardiogram (MoCG) data) and photoplethysmographic (PPG) data can be measured using a remote optical sensor such as a video camera or another imaging device. Data acquired by consumer electronic devices such as a smartphones, webcams, video cameras, gaming systems, or other imaging devices can be used in implementing systems for measuring health parameters. As a result, on-demand, non-invasive vital signs monitoring can be carried out using sensors available on existing consumer electronic devices.

Blood pressure and/or other vital signs may be measured based on continuously acquired data, without the need for cuffs, pressure points or electrodes. “Continuously” acquiring data, as used herein, means acquiring data at a sufficient frequency (e.g., a sufficient number of times per second) to allow for the derivation of the parameters described herein from that data. The data can, for example, be collected at a frequency ranging from 16 Hz to 256 Hz. In certain implementations, the data is acquired at a frequency of 128 Hz, thereby providing enough data for reliable predictive modeling.

The disclosed technology may be implemented, at least in part, using inexpensive application programs (often referred to as “apps”) executing on devices such as a smartphone, thereby empowering users to measure various health parameters on an on-demand basis. Further, automatic and potentially real-time measurement of health parameters can be leveraged to enable new applications of existing devices. For example, a data from a video camera disposed in a baby monitor can be used in determining health parameters associated with a baby and alert a parent/caregiver as needed. In another example, cameras disposed in children's wards and neonatal intensive care units (NICU) in hospitals can be used for monitoring health parameters in infants and children for who invasive measurements can be particularly difficult. Secondary parameters such as emotion, alertness level, and stress (which may be determined based on measured health parameters) can be monitored using non-contact devices such as video cameras.

The details of one or more implementations of the subject matter described in this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1A illustrates pulse transit times (PTT) using an example seismocardiogram (SCG) plot and a plot of a first derivative of corresponding photoplethysmogram (PPG) data.

FIG. 1B illustrates an example of obtaining the PTT from ballistocardiogram (BCG) and PPG data.

FIG. 2 illustrates an example of a system for contact-less blood pressure measurement.

FIG. 3 shows an example process for measuring PTT from video data.

FIG. 4 is a flowchart depicting an example of a process for computing PTT based on optical data.

FIGS. 5 and 6 are flowcharts depicting example processes of determining blood pressure based on video data.

FIG. 7 is a block diagram of a computer system that can be used in computing PTT or blood pressure from optical data.

### DETAILED DESCRIPTION

This document describes technology for determining pulse transit time (PTT) based on motioncardiogram (MoCG) data (which is related to, and generally referred to in this document as ballistocardiogram (BCG) data) and photoplethysmographic (PPG) data obtained using remote optical sensors. The optical sensors can be disposed in, for example, a video camera. The BCG is a pulsatile motion signal of the body measurable, for example, from minute movements of body parts as captured in video data featuring a subject. The PPG data can be measured, for example, by analyzing time-varying skin-tone changes of the subject as captured in the video data. The PTT thus obtained from the video can then be used to determine various health related parameters such as blood pressure.

Using technology described in this document, the health related parameters can therefore be determined without using sensors that has to be worn or even be in contact with the subject. For example, the technology described in this document allows for measurement of BCG and PPG data from video data captured by a camera (e.g., video camera, phone camera, or webcam), and therefore facilitates non-invasive measurement of health parameters, using devices or sensors located at a location remote to the subject. Further, because the technology can be implemented based on video data, cameras deployed on consumer electronic devices such as smartphones, laptops, or gaming devices can be used as sensors. This allows implementation of the technology on such third party devices, allowing the devices to be used as health monitoring equipment. For example, the technology described herein can be implemented via an application configured to execute on a smartphone, tablet, gaming device, or laptop computer, leveraging the processing power and cameras of the respective devices.

In some implementations, measurement of the PPT and/or other health parameters using this technology includes measuring BCG and/or PPG signals from optical data captured by remote sensors. The pulsatile BCG signal results from a mechanical motion of portions of the body that occurs in response to blood being pumped during a heartbeat. This motion is a mechanical reaction of the body to the internal flow of blood and can be measured, for example, by measuring video data associated with the subject. The BCG signal corresponding to a given portion of the body therefore represents the motion of the blood at that portion due to a heartbeat, but is delayed from, the heart's electrical activation (e.g. when the ventricles are electrically depolarized). In some implementations, PTT can be represented as the time taken for a pressure wave to travel between two arterial sites (e.g. from the heart to a given portion of the body) though the artery.

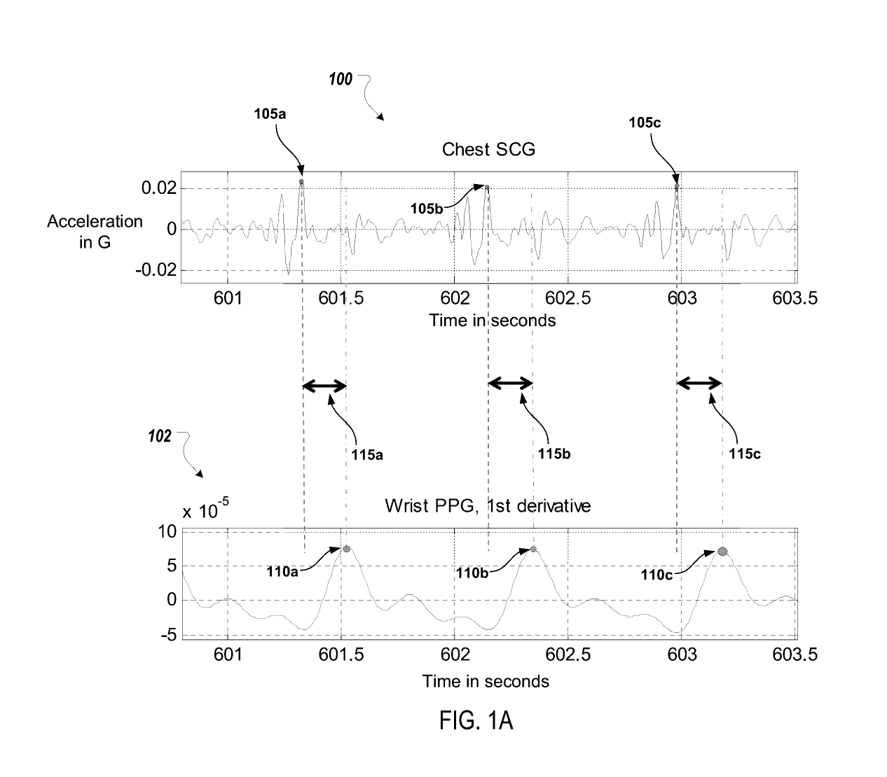

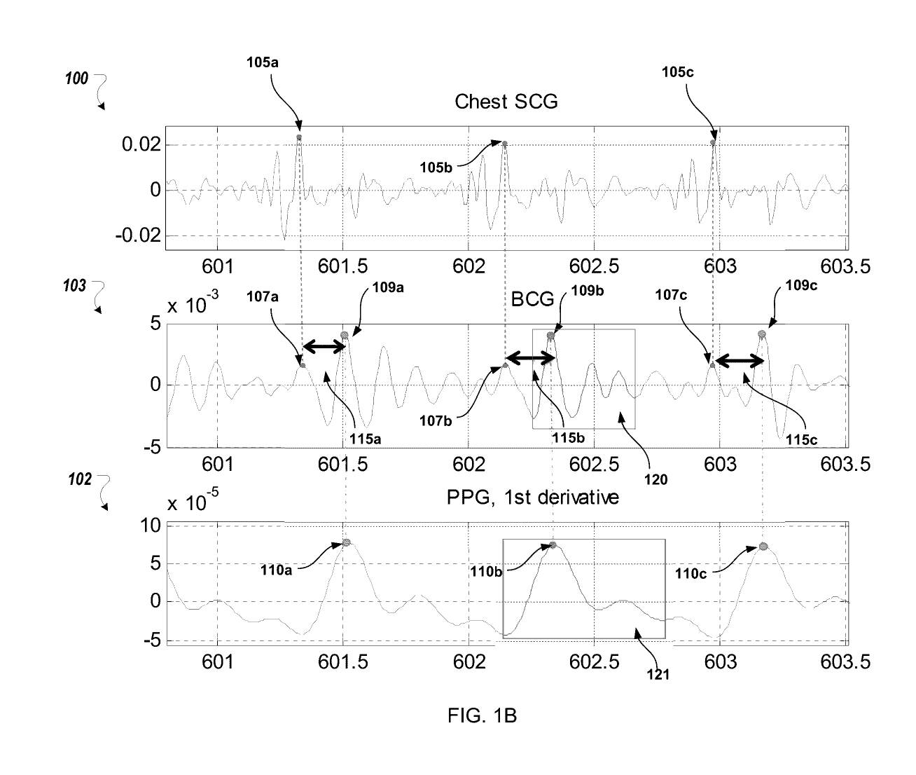

FIG. 1A illustrates pulse transit times (PTT) using an example seismocardiogram (SCG) plot 100 and a plot 102 of a first derivative of corresponding photoplethysmogram (PPG) data. The SCG plot 100 represents cardiac vibrations as measured at a location (e.g., the chest) on the body. The SCG plot 100 can be analyzed to determine points at which a pulse (or pressure wave) originates at a given location on the body. For example, the points (e.g., local maxima) 105a, 105b and 105c in the SCG plot 100 may represent time points at which a corresponding pulse originates at the chest. These points are often referred to in this document as pulse origination points 105. In some cases, the local maxima preceding the point 105 (e.g., the point 106 preceding the point 105a) can be used to represent the time point at which the corresponding pulse originates at the chest.

The time of arrival of the pulse at another location (e.g., the wrist) can be determined from PPG data obtained at the wrist. For example, the PPG data can be measured at the wrist using one or more optical sensors. Light from the optical sensors (i.e., the light sources such as LEDs of the optical sensors) is directed toward the skin of the subject, and the reflected light (which is modulated by blood volume changes underneath the skin) is measured using one or more photo-detectors (e.g., photodiodes). The output of the photo-detector may be amplified by an amplifier before being converted to a digital signal (for example, by an analog to digital converter (ADC)) that represents the PPG.

The plot 102 of FIG. 1A represents a first derivative of the PPG data, and can be used to determine the arrival time of the pulses at the wrist. For example, the local maxima 110a, 110b, and 110c (110 in general) represent the arrival times of the pulses that originated at the chest at time points represented by 105a, 105b, and 105c, respectively. These points may in general be referred to in this document as pulse arrival points 110. The plot 102 is synchronized with the SCG plot 100 such that the PTT 115 between the chest and the wrist can be determined as a time difference between the originating point at the chest and the corresponding arrival point at the wrist. In the example shown in FIG. 1A, the time difference between 105a and 110a represents the PTT 115a. Similarly, the time difference between 105b and 110b represents the PTT 115b, and the time difference between 105c and 110c represents the PTT 115c.

In some implementations, the pulse origination points 105 may be determined from BCG data measured at a given location of the body. This is illustrated in FIG. 1B, which shows a plot 103 representing BCG data measured at the wrist, the BCG data corresponding to both the SCG plot 100 and the plot 102 representing the first derivative of the PPG data. The SCG plot 100, and the plot 102 are identical to the corresponding plots shown in FIG. 1A, and are reproduced in FIG. 1B only to demonstrate how the wrist BCG data corresponds to these plots. As shown in FIG. 1B, when the plot 103 for the BCG data is aligned in time (or with respect to corresponding samples), the pulse origination point 105a substantially aligns with the local maximum 107a of the BCG plot 103. The point 107a and equivalent points in the BCG data are generally referred to herein as pulse origination points 107. The local maximum 110a and equivalent points in the PPG data are referred to herein as pulse arrival points 110. The time difference between the local maxima 107a and 110a of the BCG data and PPG data, respectively, can be used as a measure of the PTT 115a. Similarly, the time difference between the local maxima 107b and 110b can be used as a representation of the PTT 115b, and the time difference between the local maxima 107c and 110c can be used as a representation of the PTT 115c.

Described herein are techniques for identifying the pulse origination points and pulse arrival points from optical data captured by a remote sensor. For example, video data captured using a camera can be analyzed to obtain time-varying color change information (such as change in skin-tone of a subject due to subcutaneous blood flow) that represents that PPG data. The video data can also be analyzed to obtain minute time-varying motion data representing the BCG. In some implementations, this includes identifying the pulse arrival point 110 and the pulse origination point 107 from the PPG data and BCG data, respectively. In some implementations, the pulse arrival and origination points can be identified from the PPG and BCG data, respectively, based on a pattern that substantially repeats for every heartbeat. In such cases, the points can be identified by first identifying the repeated pattern in the BCG and PPG data. An example of the repeating pattern is highlighted using the box 120 in FIG. 1B. The repeating pattern can be identified in the BCG data, for example, by cross-correlating the BCG data with a template having an expected pattern. The locations of the cross-correlation peaks can then be used for detecting a presence of the repeating pattern. A repeating pattern 121 can be identified for the PPG data in a similar fashion. Once the repeating patterns are identified, the pulse origination and arrival points can be identified using the repeating patterns as the references. For example, once the repeating pattern represented by the box 120 is identified from the BCG data, the local maxima preceding the pattern can be identified as the pulse origination point 107. In some implementations, the highest peak (or local maxima) within the repeating pattern (represented by the box 121) in the PPG data is determined as a pulse arrival point.

In some implementations, the pulse origination point and/or the pulse arrival point can also be identified based on corresponding threshold conditions. For example, the pulse origination point and/or the pulse arrival point may be determined based on a threshold condition (e.g., a magnitude threshold). In some implementations, other criteria can be used as an alternative to, or in conjunction with the threshold condition, to select the pulse origination point and/or the pulse arrival points. For example, consistency in amplitude and/or location can be used to separate peaks of interest from undesirable peaks resulting from, for example, noise. This can be based on an assumption that the peaks of interest have consistent reoccurrence, as compared to noise peaks that are random.

Upon identification of the pulse arrival point 110 and the pulse origination point 107, the PTT can be determined as a time difference between the two points. In calculating PTT from digitized samples, the sampling frequency is used in determining the PTT. For example, if the pulse arrival point and the pulse origination point are separated by S samples, and if the sampling rate is F Hz, the PTT 115 can be computed as PTT=S/F seconds.

In some implementations, a determined PTT 115 may be assigned a confidence level before being used in any subsequent analysis. For example, a determined PTT 115 may be compared to the average PTT over a predetermined time range (e.g., ±10 seconds) to determine whether the determined PTT is reliable. If the given PTT differs (e.g., differs by more than a predetermined amount) from the average PTT over the predetermined time range, the given PTT may be determined to be unreliable and possibly discarded from subsequent computations. This allows for selecting reliable data points at the expense of a short latency (10 seconds in this example).

The PTT 115 can then be used to determine various health related parameters such as systolic and diastolic blood pressure as follows. The determined PTT value is related to elasticity of the blood vessels as shown in the following equation:

$$P T T = \frac{L}{P W V} = \frac{L}{\sqrt{\frac{E h}{2 ρ r}}} \qquad ( 1 )$$

where L is the vessel length, PWV is the pulse wave velocity, E is the Young's modulus, h is the vessel wall thickness, ρ is the blood density, and r is the blood vessel radius. The elasticity is in turn related to the vessel pressure P as:

E=Eₒe^{αP}, (2)

where E₀ is an elasticity parameter, and α is about 0.017 mmHg⁻¹. Based on (1) and (2), the vessel pressure P can be derived as:

P=A ln(PTT)+B (3)

where A and B are constants calculated as follows:

$$A = - \frac{2}{α}$$

$$B = \frac{1}{α} ln ( \frac{2 {L}^{2} ρ r}{{E}_{o} h} )$$

The pressure value calculated using (3) represents diastolic pressure (Dia). The systolic pressure (Sys) can then be computed as:

Sys=Dia+C*BCGₐₘₚ (4)

where A is a universal constant that applies to all users and is in units of mmHg/ms, B is an individual constant in units of mmHg, C is an individual constant in units of mmHg/mg, and BCGₐₘₚ is a measure of BCG amplitude. For example, BCGₐₘₚ can be a function of amplitude of one or both of the two BCG peaks (e.g. average of two) used in calculating the PTT. The parameters B and C for calculating the diastolic and systolic pressures may vary from one person to another. Accordingly, a process or device may need to be calibrated for an individual before use.

In some implementations, calibration can be performed, for example, based on known reference systolic and diastolic pressures (Sys_ref and Dia_ref, respectively), e.g., as input from a user or by obtaining the values from medical records. If the pressures are unknown to the user, generic values of 120/80 mmHg can be used. In such cases, the user may be allowed to alter the calibration at a later time when the actual pressures become known. By obtaining the PTT from the BCG and PPG data, as described above, the constants B and C for the particular user can be computed as follows:

B=Dia_ref−A ln(PTT), and

C=(Sys_ref−Dia_ref)/BCGₐₘₚ.

In some implementations, the calibration described above can be augmented or updated based on user-provided data. For example, a user may be asked to provide biographical data such as age, height, and weight for use in computing the calibration data. In some cases, a medical professional may measure a user's blood pressure during the calibration process. In some implementations, the calibration factors may be adjusted retroactively once the user enters valid calibration data. Calibration data may also be imported from the user's medical records if, for example, the calibration for a user is performed by his/her medical professional.

Generally, the calibrated parameters do not change frequently. These parameters may be affected, for example, by arterial diameters, arterial wall thicknesses, arterial lengths, arterial elasticity, and other physical parameters related to the cardiovascular system of a human body. The majority of the volume of blood related to PTT travels through large arteries, and is less susceptible to hydrostatic changes, temperature, or peripheral tone.

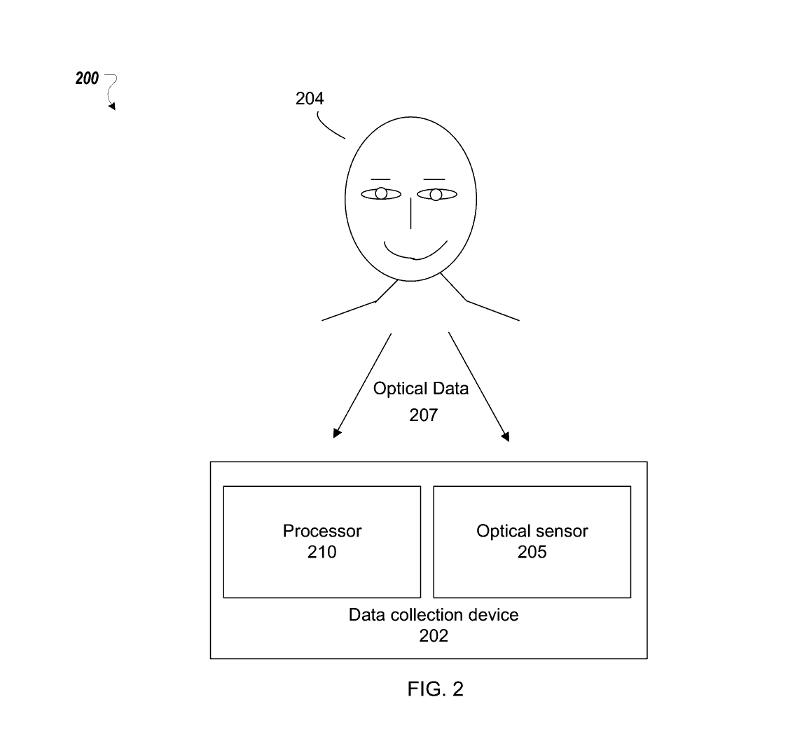

FIG. 2 illustrates an example of a system 200 that facilitates contact-less blood pressure measurement by determining PTT from optical data collected by remotely located sensors. The system includes a data collection device 202 that is located a remote or physically separated location from the subject 204. The data collection device 202 includes one or more optical sensors 205 configured to receive optical data 207 associated with the subject or patient 204. In some implementations, the data collection device 202 also includes one or more processors 210 configured to analyze the optical data collected by the optical sensors 205 to determine PTT (and by extension, other health parameters such as systolic and diastolic blood pressures) associated with the subject 204.

The data collection device 202 is configured to collect optical data related to the subject from a remote location with respect to the subject 204. For example, the data collection device 202 can be an imaging device such as a video camera that can record video frames featuring the subject 204 as the optical data 207. The spatial and temporal resolution of the video camera can be selected based on the nature of the data being recorded. For example, “high definition” digital cameras with spatial resolutions of 1280×720 pixels (720p), 1920×1080 pixels (1080p or 1080i), or 2560×1440 pixels can be used for capturing the optical data. In some implementations, cameras with higher resolution (e.g., ultra high-definition (UHD)) having spatial resolutions such as 2048×1536 pixels (2000p), 3840×2160 pixels (2160p), 4520×2540 pixels (2540p), 4096×3072 pixels (4000p), or 7680×4320 pixels (4320p) can also be used. The temporal resolution can be selected as, for example, twenty-four (or higher) frames per second.

In some implementations, the data collection device 202 can be a multi-use device that includes an imaging device. For example, the data collection device 202 can be a consumer electronic device such as a smart-phone, tablet computer, e-reader, or a laptop or desktop computer that includes an imaging device capable of capturing the optical data 207. In some implementations, the data collection device 202 can include an optical source such as one or more light emitting diode (LED) or laser generators configured to emitting an optical signal. Such optical signals can be reflected, refracted, modulated, or otherwise modified by at least a portion of the body of the subject 204, and collected by the data collection device 202 as the optical signal 207.

The data collection device 202 includes an optical sensor 205 for collecting the optical data 207. The optical sensor 205 includes electronic detectors that convert light, or a change in light, into an electronic signal. In some implementations, the optical sensor 205 includes a solid state image sensor such as a charge-coupled device (CCD) sensor or a complementary metal-oxide-semiconductor (CMOS) sensor. In some implementations where the data collection device 202 includes an optical source such as an LED, the optical sensor 205 can include an appropriate detector to measure the radiation emitted from such a source.

In some implementations, the data collection device 202 can include a transceiver that is configured to communicate wirelessly with another device to perform the functions described in this document. For example, data collected and/or computed by the data collection device 202 may be transmitted to an application executing on a mobile device or another computing device for additional analysis or storage. Various combinations of the operations described in this document may also be performed by a general purpose computing device that executes appropriate instructions encoded on a non-transitory computer readable storage device such as an optical disk, a hard disk, or a memory device.

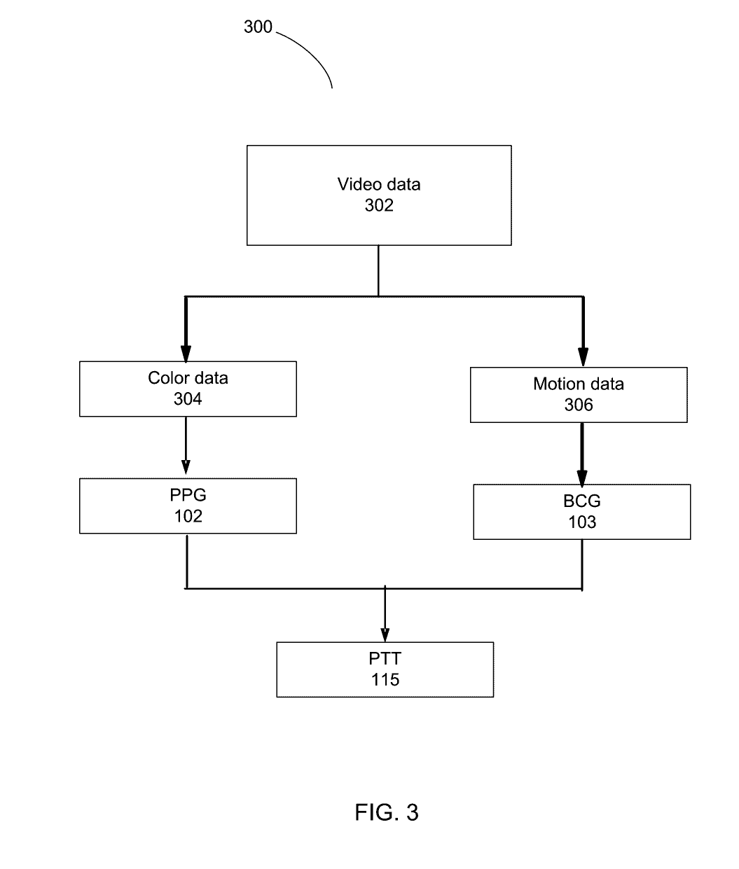

In some implementations, the data collection device 202 includes one or more processor (or processing device) 210 configured to process data collected by the optical sensor 205. For example, in implementations where the data collected by the optical sensor 205 is video data featuring a subject, the processor 210 can be configured process such video data to determine a PTT (and possibly other health parameters) associated with the subject. FIG. 3 shows an example of a process 300 for measuring PTT 115 from video data 302. The process can be performed, at least in part, by the processor 210.

In some implementations, the process 300 includes extracting color data 304 and motion data 306 from the video data 302, and obtaining PPG data 102 and BCG data 103 from the color and motion data, respectively. For example, the color data 304 can include information on a time-varying skin-tone change of the subject due to, for example, variations in subcutaneous blood flow over time. This information can be extracted, for example, by identifying, in a series of video frames, a group of one or more pixels that represents an exposed skin of the subject. The change in values of the group of pixels over the series of video frames can be indicative of the time-varying skin tone change brought about by, for example, variations in blood flow through the underlying vasculature.

In some implementations, the time-varying color change for the group of pixels is measured based on an average of values for two or more color components of the pixel. For example, if each pixel in the group has red (R), green (G), and blue (B) components, the time-varying color change of each pixel can be measured as an average (simple, or weighted) of the changes corresponding to the three different components. If the group includes more than one pixel, the time-varying color change can be measured as an average of the color changes for a plurality of pixels of the group.

In some implementations, the time-varying color change can be measured, for example, based on one or more particular colors. The color can be selected based on various criteria. In some implementations, the color can be selected based on the frequency range of the expected variations in the skin-tone change. For example, if the variations in the PPG data is expected to be in the 0.4-4 Hz range, only the green component of an RGB pixel can be selected as a representative of the overall skin-tone change. In some implementations, the color can also be selected based on characteristics of the subject. For example, a particular color component (or a weighted average of two or more particular components) may be more suitable for measuring the skin-tone change of a dark-skinned person than that of a light-skinned person. In some implementations, the color component (or combination of color components) can be selected based on the ambient light at the time of capturing the video. For example, the color component used for video taken in natural light can be different from the color component used for video taken in artificial light.

The group of pixels used for obtaining the time-varying color data 304 can be selected in various ways. In some implementations, the pixels can be selected manually, for example, by allowing a user to select pixels representing exposed skin from a particular frame of the video data 302. In some cases, such manual selection can allow for simple, yet effective, implementations, particularly where the subject is not moving with respect to the image capture device collecting the video data. In some implementations, the group of pixels can be selected automatically via image analysis techniques. For example, an automatic skin tone detection process can be used to detect pixels representing exposed skin in the frames of video data. In some implementations, the detected pixels can be further classified as particular body parts (e.g., head, arm, leg, etc.). This can allow for tracking color change data even when the subject is in motion. For example, if the subject is running on a treadmill, the pixels representing a particular body part (e.g., a leg)—which may change locations from one frame to another—can be tracked to obtain color change data at the corresponding body part.

Once the group of pixels are selected or identified, the corresponding group of pixels are tracked over a series of frames of the video data 302 to obtain the time-varying color data 304. In some implementations, the color data 304 can be processed to obtain data representing the PPG data 102. Such processing can include, for example, filtering the color data 304 in accordance with an expected frequency range of the PPG data 102. For example, the color data 304 can be processed by a filter having a passband of 0.4 Hz to 4 Hz to obtain the PPG data 102.

The process 300 also includes extracting motion data 306 from the video data 302 to obtain BCG data 103. The motion data 306 can include the minute reactionary motion of the subject's body produced due to blood flow through the body. This can be captured, for example, using a high-definition video camera capturing frames at a rate sufficient to capture the time-variations in the motion data. By identifying the location of a body part from the video data 302, and analyzing the corresponding pixels in a series of frames, motion data 306 for the body part can be obtained.

In some implementations, the motion data is obtained for a body part classified or identified in obtaining the PPG data (as described above). In some implementations, a body part is separately identified, for example, via a feature detection technique, for obtaining the corresponding motion data. In some implementations, the body part for motion data extraction is determined using a correction factor in conjunction with a body part identified in obtaining the PPG data. For example, if the color change data is calculated for pixels representing the head of the subject, a correction factor can be used to locate the pixels representing the nose, and the motion data can be extracted based on the pixels representing the nose.

The motion data 306 for a given body part can be obtained by tracking movement of corresponding pixels along a direction perpendicular to the distance between the subject and the camera (or other imaging device). For example, in the Cartesian coordinate system, if the distance between a subject and the camera is measured along the z axis (representing depth), the motion data can be captured along one or both of the x and y axes. In some implementations, the motion data can also be captured along the z axis. For example, if stereoscopic cameras capable of resolving depth information are used for capturing the video data, the motion data may be captured along the z axis.

To capture the minute motions representing the reaction of the body to blood flow, artifacts due to one or more macro motions of the subject may need to be identified and canceled. For example, if the subject is not stationary (e.g., moving with a swaying motion), the corresponding motion of the subject is identified and canceled before identifying the motion data 306 used for calculating the BCG 103. Various techniques can be used in such motion artifact cancellation. Examples of such techniques include principal component analysis (PCA), and independent component analysis (ICA). In some implementations, such motion artifact cancellation can be avoided, for example, by having the subject sit still for the duration of time the video data 302 is captured.

The motion data 306 can be represented, for example, as two-dimensional data representing movement of a particular body part as a function of time. In some implementations, the motion data 306 can be processed to obtain the BCG data 103. For example, the motion data 306 can be filtered using a digital filter to obtain the BCG data 103. The filter parameters of the digital filter can be selected, for example, based on an expected nature of the BCG signal. In some implementations, the 1-30 Hz range of the BCG signal can be expected to include most of the information needed for calculating a PTT, and accordingly a band-pass filter having a pass-band of 1-30 Hz can be selected for obtaining the BCG 103 from the motion data 306. In some implementations, a low-pass filter with a cutoff frequency around 30 Hz can also be used.

The process 300 also includes determining a PTT from the PPG data 102 and the BCG data 103. This can include synchronizing or time-aligning the PPG and BCG datasets and determining the pulse origination points and the corresponding pulse arrival points. The PTT can be calculated from the PPG 102 and the BCG 103 as described above with reference to FIGS. 1A and 1B. The PTT 115 can then be used for calculating one or more additional health parameters including, for example, systolic and diastolic blood pressure, heart rate, stroke volume, cardiac output, respiration rate, arterial stiffness, and stress. Therefore, the technology described in this document allows for calculating all such health parameters without any invasive procedure and by using a data capture device that is physically not in contact with the subject.

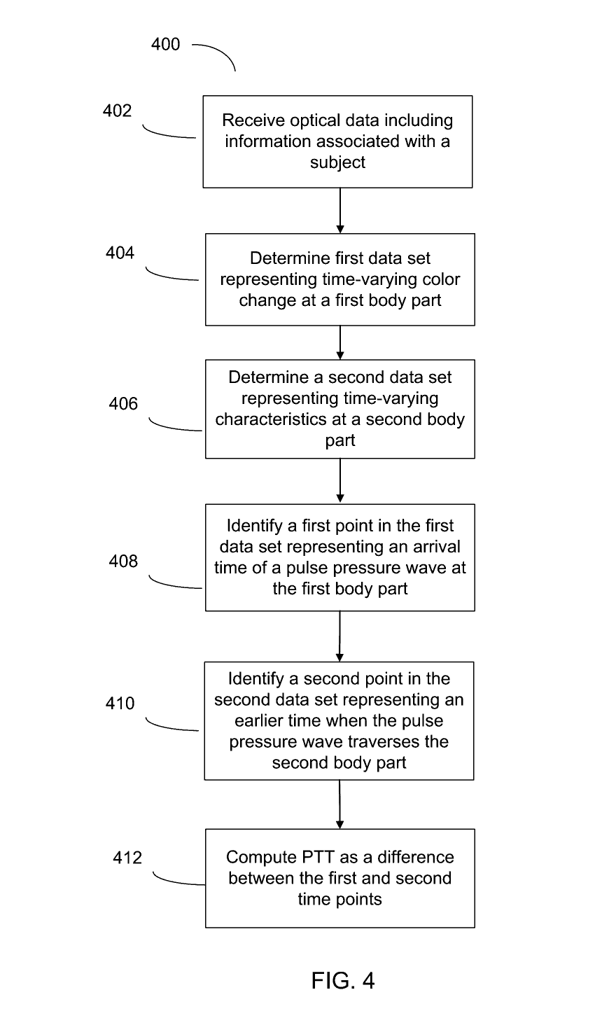

FIG. 4 is a flowchart depicting an example of a process 400 for computing PTT based on optical data. In some implementations, at least a portion of the process 400 can be performed by the processor 210 described with respect to FIG. 2. The operations of the process 400 can include receiving optical data including information associated with a subject (402). The optical data can be received from a camera or other image capture device configured to obtain data non-invasively from the subject. In some implementations, the optical data is embodied in a series of video frames featuring the subject. The optical data can also be collected by more than one image capture devices. For example, the optical data can be collected by two or more spatially separated cameras, wherein the collected optical data represents a three dimensional (3D) representation of the subject.

The operations also include determining a first data set that represents time-varying color change at a first body part of the subject (404). The first data set can be substantially similar to the color data 304 described above with respect to FIG. 3. For example, the first data set can be extracted from a series of video frames. In such cases, determining the first data set can include, for example, selecting a set of one or more pixels that represents at least a portion of the first body part, and determining a time-varying average of pixel values in the set as the first dataset. Such a time varying average can be computed based on one or more particular color components of the pixels. The one or more color components can be selected, for example, based on a nature of ambient light or a skin color of the subject. If the optical data is embodied in a series of video frames, identifying the first data set can include identifying corresponding portions in the plurality of frames, wherein the corresponding portions represent the first body part at different points in time.

Operations also include determining from the optical data, a second data set that represents time-varying characteristics at a second body part of the subject (406). The time-varying characteristics can include, for example, a motion (e.g., BCG or MoCG) at the second body part. In some implementations, the first and second body parts can be different, or can at least partially overlap with one another. Determining the second dataset can include selecting a set of one or more reference points in the optical data representing the second body part, and tracking a motion of the selected set of one or more reference points along a particular direction to determine the second dataset.

Operations also include identifying a first point in the first data set, wherein the first point represents an arrival time of a pulse pressure wave at the first body part (408). This can include, for example, computing a cross-correlation of a template segment with each of multiple segments of the first dataset, and identifying, based on the computed cross-correlations, at least one candidate segment of the first dataset as including the first point. The first point can then be identified, for example, as a local maximum or minimum, or zero crossing within the identified candidate segment.

Operations also include identifying a second point in the second data set, wherein the second point represents a time (e.g., an earlier time) when the pulse pressure wave traverses the second body part (410). The second body part can be different from the first body part, or can include at least a portion of the first body part. For example, the first and second body parts can be the forehead and neck, respectively. The first and second data sets may be aligned in time before identifying the second point. In some implementations, identifying the second point can include computing a cross-correlation of a template segment with each of multiple segments of the second dataset, and identifying, based on the computed cross-correlations, at least one candidate segment of the second dataset as including the second point. The second point can then be identified within the second segment, for example, as a local maximum, local minimum, or zero crossing point.

The operations also includes computing PTT as a difference between the first and second time points (412). The PTT thus computed can then be used in determining additional health parameters including, for example, diastolic and systolic blood pressures, and stroke volume. For example, the diastolic pressure can be calculated as a function of the logarithm of the PTT, and the systolic pressure can be calculated as a function of the diastolic pressure.

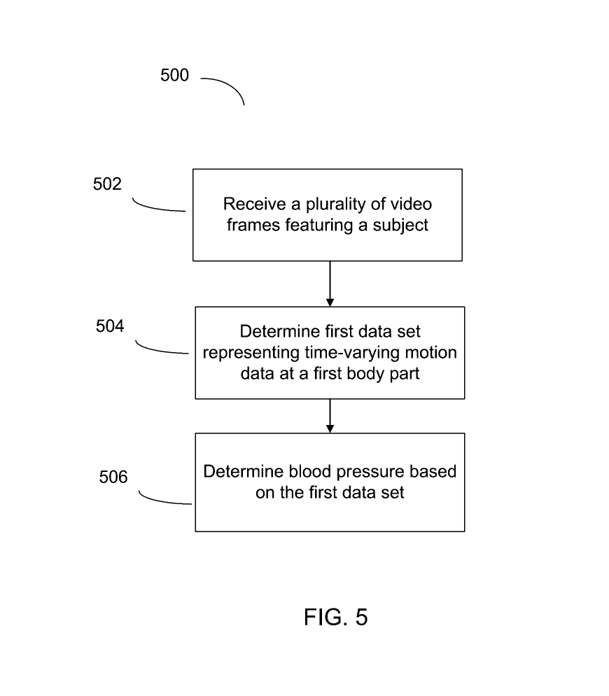

FIG. 5 is a flowchart depicting an example process 500 for determining blood pressure based on video data. In some implementations, at least a portion of the process 500 can be performed by the processor 210 described with respect to FIG. 2. The operations of the process 500 can include receiving a plurality of video frames featuring a subject (502). The operations also include determining a first data set representing time-varying motion data at a first body part (504), and determining blood pressure based on the first data set (506). In some implementations, the blood pressure is determined as a function of PTT computed for the subject. In some implementations, computing the PTT can include determining, from the video data, a second dataset representing time-varying color change at the first body part of the subject, and identifying a first point in the second data set. The first point represents an arrival time of the pulse pressure wave at the first body part of the subject. Computing the PTT also includes identifying a second point in the first data set. The second point represents an earlier time at which the pulse pressure wave traverses the second body part of the subject. The PTT can then be determined as a difference between the first and second time points.

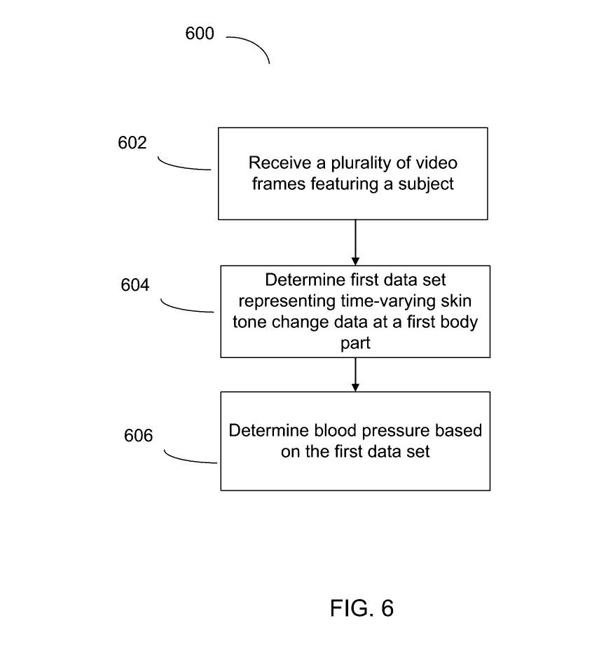

FIG. 6 is a flowchart depicting an example process 600 for determining blood pressure based on video data. In some implementations, at least a portion of the process 600 can be performed by the processor 210 described with respect to FIG. 2. The operations of the process 600 can include receiving a plurality of video frames featuring a subject (602). The operations also include determining a first data set representing time-varying skin tone change data at a first body part (604), and determining blood pressure based on the first data set (506). In some implementations, the blood pressure is determined as a function of PTT computed for the subject. In some implementations, computing the PTT can include determining, from the video data, a second dataset representing time-varying motion at the first body part of the subject, and identifying a first point in the first data set. The first point represents an arrival time of the pulse pressure wave at the first body part of the subject. Computing the PTT also includes identifying a second point in the second data set. The second point represents an earlier time at which the pulse pressure wave traverses the second body part of the subject. The PTT can then be determined as a difference between the first and second time points.

The PTT and blood pressure measured non-invasively using the technology described above can be used in determining various other health-related parameters, and in various applications. Some examples of such health related parameters and applications are discussed below.

### Heart Rate, Stroke Volume, and Cardiac Output

Motion data such as the BCG is typically periodic with respect to heartbeats, and the heart rate information can be obtained from such data. A stroke volume can be calculated from the amplitudes of one or both peaks or local maxima used in calculating the PTT. The cardiac output can then be calculated, for example, as a product of the heart rate and the stroke volume.

### Detection of Arterial Stiffness

Another health-related characteristic that can be detected using the technology described herein is arterial stiffness, which is an indicator for vascular health (e.g. arteriosclerosis), risk for hypertension, stroke, and heart attack. The stiffer the arteries, the faster the blood wave travels (due to fluid dynamics) and thus the shorter the PTT. The processor can therefore be programmed to calculate arterial stiffness as a function of the pulse transit time (PTT). The arterial stiffness value can be used as one of multiple factors for assessing the overall health of the user. In some cases, for example, the arterial stiffness of the user can be used to determine a health score for the user.

The arterial stiffness of a subject tends to decrease as the activity level of the subject (e.g., the number of times per week that the subject exercises) increases. Thus, the calculated arterial stiffness can be used to track the progress of a subject involved in an exercise regimen. This can serve as positive feedback for the user in addition to conventional feedback, such as weight loss.

### Fitness-Related Applications

The technology described herein can be incorporated into various fitness applications that allow the user to monitor his or her fitness level. As an example, video data captured for a subject on an exercise machine can be analyzed to determine the total number of steps taken by the user during that time. In addition, the number of calories burned over a given period of time can be determined by analyzing the activity level of the user and the heart rate of the user. Using both the activity level and the heart rate to determine calories burned can lead to a more accurate estimation of caloric output.

### Monitoring Stress Levels

In some implementations, the technology described herein can also be used to determine the stress level of a user. For example, one or more of heart rate, heart rate variability (HRV), and blood pressure (BP) can be used as indicators of stress. Specifically, the values of these parameters increase as stress levels increase. Thus, by comparing these values to baseline values of the user for associated parameters, the level of stress of the user can be estimated. The stress level can, for example, be provided to the user as a stress score. In some implementations, by analyzing a video feed received from a camera disposed in the cabin of a vehicle, a driver can be prevented from operating the vehicle if his/her stress level is determined to be higher than a threshold level. This can help, for example, reduce occurrences of stress-related traffic issues (e.g., road rage) and accidents.

### Triage Applications

The technology described above can also be used to assist triage medical personnel in various settings. For example, various health parameters for patients in an emergency room waiting area can be determined from a video feed of the waiting area to prioritize medical care. As a result, patients in need of urgent treatment may be treated ahead of patients with less threatening conditions. The technology can also be used in prioritizing medical care at the scene of an accident or another emergency situation. For example, an initial assessment of health parameters of multiple victims may be obtained in parallel by analyzing a video feed of the scene of the accident. The medical personnel can accordingly prioritize to focus their efforts on victims in more urgent need of medical care. While doing so, the vital signs of those victims who were initially assessed may be monitored and transmitted to a central monitoring station. Thus, in the event that the condition of one of those victims being monitored deteriorates to the point of requiring urgent medical attention, medical personnel in the area can be directed to that victim to provide the necessary medical care.

In addition to being used in the triage context, the technology described herein may be used to assist medical personnel in a hospital setting. Once a patient is stabilized following triage, he or she is typically monitored based on a provider's standard of care or mandate (e.g., according to an accountable care organization (ACO)). In some implementations, the vital signs of the patient can be monitored, for example, via a video feed, outside of the triage context to ensure that the care that the patient is receiving is appropriate in view of the patient's vitals. A provider's standard of care may require a patient to go through a progression of steps before the patient is deemed to be ready for discharge. The technology described herein can be used to monitor the vital signs of the patient non-invasively during each step of the progression.

### First Responder and Military Applications

The technology described herein can be used to non-invasively monitor health parameters of first responders such as firefighters and police offers, and military personnel such as air force pilots and tank drivers. The vital signs of such personnel may be monitored before, during, and after any stressful events that they experience to ensure that they receive the help they need. This can be done, for example, by analyzing video feed from a camera positioned in a helmet, a police cruiser, airplane, or tank.

### Alertness Monitoring

In some implementations, the technology described herein can be used for monitoring the alertness of one or more users. This can be particularly advantageous for personnel who perform tasks that require a significant amount of attention and concentration. Examples of such personnel include air traffic controllers, pilots, military truck drivers, tanker drivers, security guards, TSA agents, intelligence analysts, etc.

To monitor the alertness of the user, one or more of the heart rate, blood pressure, and activity level of the user can be analyzed. Each of these parameters tends to decrease as a subject becomes less alert. Thus, when one or more of the monitored parameters falls a predetermined amount from the corresponding baselines, a determination can be made that the user's alertness level has dropped to an unacceptable level. In some implementations, upon determining that the user's alertness level has dropped to an unacceptable level, an alarm or another form of instant communication may be initiated to raise the alertness level of the user and thus reduce risk of harm to the user and others.

### Prediction of Medical Events

While certain examples discussed above relate to the use of skin tone change data and motion data (e.g., MoCG data) to diagnose medical conditions or events that were already experienced by the user, in certain implementations, the processor can be programmed to use this data to predict medical conditions before they happen. For example, the heart rate, heart rate variability, and blood pressure of the wearer can be monitored and processed by the processor to make such predictions. One example of a medical event that can be predicted in a subject is tachycardia. Tachycardia is when a subject's heart rate is over 100 beats per minute. If a subject's heart rate is trending upwards, a prediction can be made as to when the subject will experience tachycardia. Other examples of medical events that can be predicted are hypertension and stroke. For example, if a subject's blood pressure is increasing over time (e.g., if the rate of change of the blood pressure is above a threshold), a prediction can be made as to when the subject will experience hypertension. Hypertension is diagnosed when a subject's blood pressure exceeds 140/90 mmHg. If the increase is rapid, a prediction can be made as to when the subject will have a high likelihood of experiencing a stroke. Similarly, if a subject's blood pressure is decreasing rapidly (e.g., if the rate of change of the blood pressure is negative and below a threshold), a prediction can be made as to whether the subject will have a heart condition.

In cases where the heart rate variability of the subject is used to predict a medical event, whether the subject experiences arrhythmia (e.g., atrial fibrillation) can determine what an appropriate heart rate variability of the subject is. For example, a subject who experiences arrhythmia may have a high heart rate variability, but this may be normal given the subject's condition.

### Medication Compliance

The technology described herein can also be used to non-invasively monitor whether a patient is adhering to a prescribed medication regimen. For example, the non-invasively measured blood pressure data can be used in warning or reminding a patient to take his or her medication. The technology can be used in this manner to monitor adherence to a prescribed medication schedule for any of various other medications that impact the various different vital signs that can be non-invasively measured using the technology described above.

Connectivity with Other Devices

In some implementations, the data collection device 202 can be configured to communicate with other computing devices. For example, the device 202 can include a transceiver module that can send data to, and receive data from, a server computer. In such cases, the device 202 can be configured to act as a client within a client-server architecture. Implementations in which the device 202 is a mobile device such as a smartphone, the device 202 may communicate the collected information to the server computer over the Internet. The server computer can be configured to receive and store data provided by the device 202 and share the data with other computing devices. For example, a hospital, nursing home, or elder-care center may use a server computer (or another central computer acting as a hub) that is configured to receive communications from devices 202 monitoring patients or residents. In such cases, the server computer can be configured to determine, based on data received from a particular device 202, that a patient or user being monitored by the device 202 is in need of assistance. The server computer can be configured to alert appropriate personnel accordingly. For example, based on data (e.g., heart rate or blood pressure) received from a particular device 202, the server computer may determine that the user of the particular device is experiencing (or is likely to experience) a health-related emergency, and alert appropriate caregivers automatically (e.g., by sending a text message or paging message to the caregivers, triggering an alarm, or initiating an emergency call). In some implementations, the device 202 itself may make such a determination and forward the information to the server computer for taking an appropriate action.

In some implementations, the device 202 can be configured to communicate over a network (e.g., a Wi-Fi network) with other devices connected to the network. For example, the device 202 can be configured to communicate with a Wi-Fi enabled thermostat to facilitate control of ambient temperature based on vital signs data collected by the device 202. For example, temperature data collected using the device 202 can be used to determine that the user is cold, and the ambient temperature can be increased accordingly.

In some implementations, the device 202 can be implemented as a part of a gaming device such as a video game console, or configured to communicate with the gaming device. In such cases, data from the device 202 can be used to control the gaming device based on an identity and/or state of the body of the user. For example, blood pressure data and/or heart rate obtained using the device 202 can be used to determine an interest level or engagement level of the user. If the user is determined to show more interest in certain game situations as opposed to others, the gaming device can be configured to adaptively provide game situations that the user is interested in. If the data from the device 202 indicates a low level of interest, steps can be taken (e.g. increasing the background sound level, playing a stimulating track, or introducing additional challenges) to increase the interest level of the user. This way, games being played on the gaming device can be made more appealing to the user. In some implementations, the gaming device can be configured to be turned off if the user's body state is determined to be in a potentially harmful condition. For example, if the blood pressure or heart rate data from the device 202 indicates that the stress level of the user is above a threshold, the gaming device can be instructed to shut down to prevent the user from continuing to play.

In some implementations, the device 202 can be configured to communicate with a transceiver module in a vehicle. In such cases, the transceiver module of the vehicle can be configured to provide feedback to other modules in the vehicle based on data received from the device 202 (either directly, or via a server). For example, the transceiver module of the car can be configured to provide feedback signals to a temperature control system of the vehicle to adjust the temperature based on vital signs data received from the device 202. In another example, the transceiver module may use data from the device 202 to provide feedback to a collision avoidance system that, for example, triggers an alarm (and/or slows the vehicle down) upon determining that a driver is not adequately alert.

### Computing Device

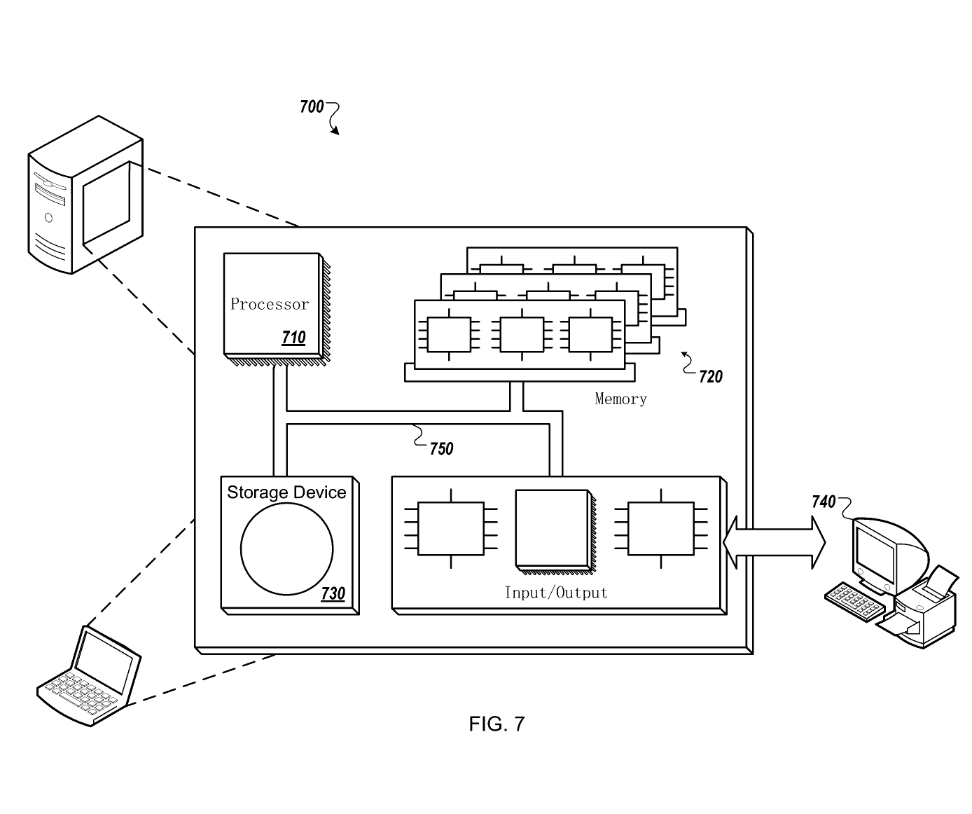

FIG. 7 is block diagram of an example computer system 700 that can be used for performing one or more operations related to the technology described above. In some implementations, the computer system 700 can be used to implement any portion, module, unit or subunit of the device 202, or computing devices and processors referenced above. The system 700 includes a processor 710, a memory 720, a storage device 730, and an input/output device 740. Each of the components 710, 720, 730, and 740 can be interconnected, for example, using a system bus 750. The processor 710 is capable of processing instructions for execution within the system 700. In one implementation, the processor 710 is a single-threaded processor. In another implementation, the processor 710 is a multi-threaded processor. The processor 710 is capable of processing instructions stored in the memory 720 or on the storage device 730.

The memory 720 stores information within the system 700. In one implementation, the memory 720 is a computer-readable storage device that includes a non-transitory computer readable medium. In general, non-transitory computer readable medium is a tangible storage medium for storing computer readable instructions and/or data. In some cases, the storage medium can be configured such that stored instructions or data are erased or replaced by new instructions and/or data. Examples of such non-transitory computer readable medium include a hard disk, solid-state storage device, magnetic memory or an optical disk. In one implementation, the memory 720 is a volatile memory unit. In another implementation, the memory 720 is a non-volatile memory unit.

The storage device 730 is capable of providing mass storage for the system 700. In one implementation, the storage device 730 is a computer-readable medium. In various different implementations, the storage device 730 can include, for example, a hard disk device, an optical disk device, or some other large capacity storage device.

The input/output device 740 provides input/output operations for the system 700. In one implementation, the input/output device 740 can include one or more of a network interface devices, e.g., an Ethernet card, a serial communication device, e.g., an RS-232 port, and/or a wireless interface device, e.g., and 802.11 card. In another implementation, the input/output device can include driver devices configured to receive input data and send output data to other input/output devices, e.g., keyboard, printer and display devices.

Although an example processing system has been described in FIG. 7, implementations of the subject matter and the functional operations described in this specification can be implemented in other types of digital electronic circuitry, or in computer software, firmware, or hardware, including the structures disclosed in this specification and their structural equivalents, or in combinations of one or more of them. Implementations of the subject matter described in this specification can be implemented as one or more computer program products, i.e., one or more modules of computer program instructions encoded on a tangible program carrier, for example a computer-readable medium, for execution by, or to control the operation of, a processing system. The computer readable medium can be a machine-readable storage device, a machine-readable storage substrate, a memory device, or a combination of one or more of them.

The term “processing system” encompasses all apparatus, devices, and machines for processing data, including by way of example a programmable processor, a computer, or multiple processors or computers. The processing system can include, in addition to hardware, code that creates an execution environment for the computer program in question, e.g., code that constitutes processor firmware, a protocol stack, a database management system, an operating system, or a combination of one or more of them.

A computer program (also known as a program, software, software application, script, or code) can be written in any form of programming language, including compiled or interpreted languages, or declarative or procedural languages, and it can be deployed in any form, including as a stand-alone program, a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

Computer readable media suitable for storing computer program instructions and data include all forms of non-volatile memory, media and memory devices, including by way of example, semiconductor memory devices, e.g., EPROM, EEPROM, and flash memory devices; magnetic disks, e.g., internal hard disks or removable disks; magneto optical disks; and CD ROM and DVD ROM disks. The processor and the memory can be supplemented by, or incorporated in, special purpose logic circuitry.

Implementations of the subject matter described in this specification can be implemented in a computing system that includes a back end component, e.g., a data server, or that includes a middleware component, e.g., an application server, or that includes a front end component, e.g., a client computer having a graphical user interface or a Web browser through which a user can interact with an implementation of the subject matter described is this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), e.g., the Internet.

The computing system can include clients and servers. A client and server are generally remote from each other and typically interact through a communication network. The relationship of client and server arises by virtue of computer programs running on the respective computers and having a client server relationship to each other.

While this specification contains many specific implementation details, these should not be construed as limitations on the scope of what may be claimed, but rather as descriptions of features that may be specific to particular implementations. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can, in some cases, be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

A number of implementations of the invention have been described. Nevertheless, it will be understood that various modifications may be made without departing from the spirit and scope of the technology described in this document. Accordingly, other implementations are within the scope of the following claims.

1. A computer-implemented method comprising:
receiving, at one or more processing devices, optical data including information associated with a subject;
determining, by the one or more processing devices from the optical data, a first dataset representing time-varying color change at a first body part of the subject;
determining, by the one or more processing devices from the optical data, a second dataset representing time-varying characteristics at a second body part of the subject;
identifying, by the one or more processing devices, a first point in the first dataset, the first point representing a time at which a pulse pressure wave traverses the first body part of the subject;
identifying, by the one or more processing devices, a second point in the second dataset, the second point representing a time at which the pulse pressure wave traverses the second body part of the subject; and
computing a pulse transit time (PTT) as a difference between the first and second points, the PTT representing a time taken by the pulse pressure wave to travel from the second body part to the first body part of the subject.

2. The method of claim 1, wherein the second body part comprises at least a portion of the first body part.

3. The method of claim 1, wherein the time varying characteristics comprises at least one of: motion of the second body part and time-varying color change.

4. (canceled)

5. The method of claim 1, wherein the optical data comprises video data that includes a plurality of frames featuring the subject.

6. The method of claim 5, further comprising identifying corresponding portions in the plurality of frames, wherein the corresponding portions represent the first body part at different points in time.

7. (canceled)

8. The method of claim 5, wherein determining the first dataset comprises:
selecting from the video data, a set of one or more pixels that represents at least a portion of the first body part; and
determining the first dataset as a time-varying average of pixel values in the set.

9. (canceled)

10. The method of claim 8, wherein the time-varying average is computed based on a particular color component of the pixel values in the set.

11. The method of claim 10, wherein the color component is selected based on at least one of: a nature of ambient light in which the video data is captured and a nature of skin color of the subject.

12. (canceled)

13. The method of claim 1, wherein determining the second dataset comprises:
selecting a set of one or more reference points in the optical data representing the second body part; and
tracking a motion of the selected set of one or more reference points along a particular direction to determine the second dataset.

14. The method of claim 1, wherein the second dataset comprises ballistocardiogram (BCG) data.

15. (canceled)

16. (canceled)

17. The method of claim 1, wherein identifying the first point comprises:
computing a cross-correlation of a template segment with each of multiple segments of the first dataset;
identifying, based on the computed cross-correlations, at least one candidate segment of the first dataset as including the first point; and
identifying a first local maximum or minimum, or zero-crossing within the identified candidate segment as the first point.

18. The method of claim 1, wherein identifying the second point comprises:
computing a cross-correlation of a template segment with each of multiple segments of the second dataset;
identifying, based on the computed cross-correlations, at least one candidate segment of the second dataset as including the second point; and
identifying a first local maximum or minimum, or zero-crossing within the identified candidate segment as the second point.

19. The method of claim 1, further comprising computing a blood pressure of the subject as a function of the PTT.

20.-22. (canceled)

23. The method of claim 5, wherein determining the first or second dataset comprises identifying at least one of the first body part and the second body part from the video data.

24.-30. (canceled)

31. A system comprising:
memory; and
one or more processing devices configured to:
receive optical data including information associated with a subject,
determine, from the optical data, a first dataset representing time-varying color change at a first body part of the subject,
determine, from the optical data, a second dataset representing time-varying characteristics at a second body part of the subject,
identify a first point in the first dataset, the first point representing a time at which a pulse pressure wave traverses the first body part of the subject,
identify a second point in the second dataset, the second point representing a time at which the pulse pressure wave traverses the second body part of the subject, and
compute a pulse transit time (PTT) as a difference between the first and second points, the PTT representing a time taken by the pulse pressure wave to travel from the second body part to the first body part of the subject.

32. The system of claim 31, wherein the second body part comprises at least a portion of the first body part.

33. The system of claim 31, wherein the time varying characteristics comprises at least one of: motion of the second body part and time-varying color change.

34. (canceled)

35. The system of claim 31, wherein the optical data comprises video data that includes a plurality of frames featuring the subject.

36. The system of claim 35, where in the one or more processing devices are further configured to identify corresponding portions in the plurality of frames, wherein the corresponding portions represent the first body part at different points in time.

37. (canceled)

38. The system of claim 35, wherein determining the first dataset comprises:
selecting from the video data, a set of one or more pixels that represents at least a portion of the first body part; and
determining the first dataset as a time-varying average of pixel values in the set.

39. (canceled)

40. The system of claim 38, wherein the time-varying average is computed based on a particular color component of the pixel values in the set.

41. The system of claim 40, wherein the color component is selected based on at least one of: a nature of ambient light in which the video data is captured and a nature of skin color of the subject.

42. (canceled)

43. The system of claim 31, wherein determining the second dataset comprises:
selecting a set of one or more reference points in the optical data representing the second body part; and
tracking a motion of the selected set of one or more reference points along a particular direction to determine the second dataset.

44. The system of claim 31, wherein the second dataset data comprises ballistocardiogram (BCG) data.

45.-46. (canceled)

47. The system of claim 31, wherein identifying the first point comprises:
computing a cross-correlation of a template segment with each of multiple segments of the first dataset;
identifying, based on the computed cross-correlations, at least one candidate segment of the first dataset as including the first point; and
identifying a first local maximum or minimum, or zero-crossing within the identified candidate segment as the first point.

48. The system of claim 31, wherein identifying the second point comprises:
computing a cross-correlation of a template segment with each of multiple segments of the second dataset;
identifying, based on the computed cross-correlations, at least one candidate segment of the second dataset as including the second point; and
identifying a first local maximum or minimum, or zero-crossing within the identified candidate segment as the second point.

49. The system of claim 31, wherein the one or more processors are configured to compute a blood pressure of the subject as a function of the PTT.

50.-52. (canceled)

53. The system of claim 35, wherein determining the first or second dataset comprises identifying at least one of the first body part and the second body part from the video data.

54.-60. (canceled)

61. One or more machine-readable storage devices storing instructions that, upon execution by one or more processing devices, cause the one or more processing devices to perform operations comprising:
receiving optical data including information associated with a subject;
determining from the optical data, a first dataset representing time-varying color change at a first body part of the subject;
determining from the optical data, a second dataset representing time-varying characteristics at a second body part of the subject;
identifying a first point in the first dataset, the first point representing a time at which a pulse pressure wave traverses the first body part of the subject;
identifying a second point in the second dataset, the second point representing a time at which the pulse pressure wave traverses the second body part of the subject; and
computing a pulse transit time (PTT) as a difference between the first and second points, the PTT representing a time taken by the pulse pressure wave to travel from the second body part to the first body part of the subject.

62.-67. (canceled)
